(19) United States
(12) Patent Application Publication
(10) Pub. No.: US 2026/0341504 A1
(43) Pub. Date: Oct. 29, 2026

(54) SYSTEM IMPLEMENTED IN A NEURAL NETWORK FOR DETECTING BLOOD VESSELS BY MEANS OF PIXEL SEGMENTATION

(71) Applicants: CLÍNICA OFTALMOLÓGICA DEL CARIBE S.A.S., Barranquilla (CO); UNIVERSIDAD SIMÓN BOLÍVAR, Barranquilla, Atlántico (CO)

(72) Inventors: Reynaldo VILLARREAL GONZÁLEZ, Barranquilla, Atlántico (CO); Juan PESTANA NOBLES, Barranquilla, Atlántico (CO); Roberto PESTANA, Barranquilla, Atlántico (CO); Paola AMAR SEPÚLVEDA, Barranquilla, Atlántico (CO); Sonia ESCAF, Barranquilla, Atlántico (CO); Luis José ESCAF JARABA, Barranquilla, Atlántico (CO); Jorge José MARTÍNEZ RAMÍREZ; Fanny Judith SALES PUCCINI, Barranquilla, Atlántico (CO)

(21) Appl. No.: 19/104,089

(22) PCT Filed: Aug. 16, 2023
(86) PCT No.: PCT/CO2023/000016

§ 371 Date: Feb. 14, 2025

(30) Foreign Application Priority Data
Aug. 17, 2022 (CO) NC2022/0011610

(51) Int. Cl. G16H 50/20

(52) U.S. Cl. CPC G16H 50/20

(57) ABSTRACT

The proposed solution addresses the problem by providing a novel method that utilizes a pre-trained artificial intelligence (AI) model. The AI processes images through pixel clustering to identify and differentiate the tones of pixels. This enables segmentation and differentiation of blood vessels from the rest of the eye fundus.

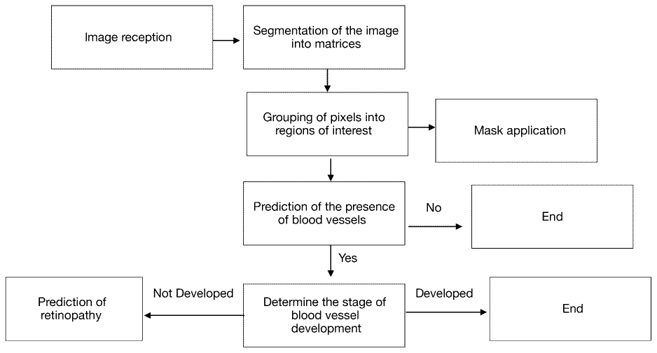

### FIELD OF THE INVENTION

The present invention belongs to the field of healthcare, particularly in optics related to the measurement and detection of ocular blood vessels. The proposed process is specifically implemented through automated methods using machine learning.

### STATE OF THE ART

The patent CN111144413 ‘Iris Positioning Method and Computer-Readable Storage System’ was found. This invention discloses a method for iris positioning using an infrared image of a human face or eyes. The method applies pixel clustering, Hough transform, and segmentation techniques to detect and process the iris area.

Additionally, US2021259546 ‘Portable System for Identifying Potential Cases of Diabetic Macular Edema Using Image Processing and Artificial Intelligence’ was identified. This invention describes a portable system for detecting diabetic macular edema using a portable ophthalmoscope, which captures an eye fundus image. The image is transmitted to an embedded system that processes it using artificial intelligence to detect the condition.

Another relevant patent, CN105118049, discloses a ‘Superpixel-Based Image Segmentation Method’ that applies clustering techniques for image segmentation.

None of these solutions provide the technical advantages or specific features of the proposed invention. Unlike existing methods, this invention does not rely solely on pixel clustering but integrates this method with discriminatory and classification algorithms to diagnose retinal diseases, specifically retinopathy.

### BRIEF DESCRIPTION OF THE INVENTION

The proposed solution addresses the problem by providing a novel method that utilizes a pre-trained artificial intelligence (AI) model. The AI processes images through pixel clustering to identify and differentiate the tones of pixels representing blood vessels. This enables the segmentation and distinction of blood vessels from the rest of the eye fundus.

### DETAILED DESCRIPTION OF THE INVENTION

The measurement of blood vessel proliferation for diagnosing retinopathy in newborns traditionally involves mechanical methods interpreted by medical specialists. These methods pose challenges in remote locations with limited healthcare infrastructure and require expensive specialized devices.

Retinopathy diagnosis in newborns is determined by the presence of blood vessels in different retinal zones. The retina is divided into three zones, with Zone 1 being the innermost, located in the macula, followed by Zones 2 and 3.

The proposed method leverages artificial intelligence for image processing, enabling the detection of blood vessels and their extension. This eliminates the need for manual observation, allowing remote diagnostics.

The AI-based method requires capturing an image, which, in a preferred embodiment, is taken with a 160° wide-angle camera for adults and a 120° camera for premature infants, with a resolution of 1080p. The AI consists of an input for normalized image data, eight hidden processing layers, and an output indicating the presence and extent of blood vessels.

For AI training, image datasets are used with predefined matrices of identical dimensions. A color mask is applied to highlight specific pixels, helping the AI learn to differentiate blood vessels from other structures.

The AI processes a digitally captured image by transforming it into three pixel-value matrices for machine understanding. These matrices are standardized using a normalization filter, resizing them to 576×576 pixels to match training data.

The AI employs an eight-layer deep neural network:
The first four hidden layers generate weights to classify pixels as vessels or non-vessels using linear regression and a ReLU activation function.
The remaining four dropout layers prevent overfitting due to pixel variation, also using ReLU activation.
The final output node uses a Sigmoid activation function to determine whether the detected structure is a blood vessel.

Additionally, the algorithm determines the stage of vessel development based on clustered positive pixels, helping diagnose retinopathy progression.

1. Artificial intelligence process applied to a neural network for the identification and measurement of ocular blood vessels, characterized by comprising a multilayer neural network, pre-fed with an input layer, at least eight hidden layers, and an output layer within an integrated development environment.

2. Artificial intelligence process according to claim 1, wherein the input layer corresponds to the color palette of the pixel referenced as positive, obtained from the image.

3. Artificial intelligence process according to claim 1, wherein the hidden layers were trained so that the first four hidden layers use a linear regression algorithm complemented with a ReLU activation function, and the second four hidden layers are dropout layers with a ReLU activation function.

4. Artificial intelligence process according to claim 1, wherein the input layer processes the image by segmenting it into three matrices: the first matrix comprises the original image, the second matrix comprises the image segmented by pixels of interest, and the third matrix comprises the pixels of interest grouped into regions.

5. Artificial intelligence process according to claim 1, wherein the grouping of pixels into regions of interest is performed by applying a mask.

6. Artificial intelligence process according to claim 1, wherein the output layer, using a sigmoid function, delivers a prediction of the presence or absence of blood vessels in the eye fundus.

7. Artificial intelligence process according to claim 1, wherein, if the output layer identifies the presence of blood vessels in the eye fundus, it determines their stage of development based on their pixel extension.
